(19) United States
(12) Patent Application Publication
(10) Pub. No.: US 2021/0106700 A1
(43) Pub. Date: Apr. 15, 2021

(54) Fractionated Dosing of a Phospholipid Ether Analog for the Treatment of Cancer

(71) Applicant: Cellectar Biosciences, Inc., Madison, WI (US)

(72) Inventor: Jarrod Longcor, East Haven, CT (US)

(21) Appl. No.: 17/044,752

(22) PCT Filed: Apr. 10, 2019
(86) PCT No.: PCT/US19/26828

§ 371 Date: Oct. 1, 2020

Related U.S. Application Data
(60) Provisional application No. 62/655,615, filed on Apr. 10, 2018.

(51) Int. Cl. A61K 51/04; A61N 5/10; A61P 35/00

(52) U.S. Cl. CPC A61K 51/0408; A61P 35/00; A61N 5/1077

(57) ABSTRACT

Disclosed herein is a fractionated dosing regimen of ¹³¹I-labeled 18-(p-iodo-phenyl)octadecyl phosphocholine for the treatment of cancer.

### CROSS REFERENCE TO RELATED APPLICATIONS

This application claims priority to U.S. Provisional Patent Application No. 62/655,615, filed on Apr. 10, 2018, the entire contents of which are fully incorporated herein by reference.

### TECHNICAL FIELD

The present disclosure is directed to the use of radiolabeled phospholipid analogs in specific dosing cycles for the treatment of cancer.

### BACKGROUND

Selective retention of radiolabeled phospholipid ether analogs in a variety of model systems has been constrained by issues related to the relatively rapid clearance of the radiopharmaceutical compound and/or accumulation of the compound in non-target tissues. Non-target tissue uptake can decrease the efficacy of radiopharmaceutical treatment by, for example, causing excessive exposure of radiosensitive tissues to the administered radioactivity.

There remains a significant need in the art for radiopharmaceuticals that exhibit a rapid clearance from non-target tissues as well as an extended half-life in the plasma, while still retaining its specificity and avidity for neoplastic tissue. Such an agent should serve as a carrier for a cytotoxic agent for site-specific eradication of malignant tumor tissue.

Currently, there are few chemical compounds that preferentially target cancer cells. One such compound is CLR1404. Generally, CLR1404 is a promising new tumor-selective diagnostic imaging agent used to monitor the treatment response of several tumor treatment modalities. Radioiodinated CLR1404, a second-generation phospholipid ether (“PLE”) analog with the following structure.

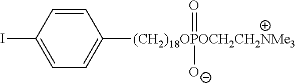

has displayed remarkable tumor selectivity in 55/60 xenograft, orthotopic and transgenic cancer and cancer stem cell derived animal models making the core molecule an ideal platform for an anti-cancer drug delivery vehicle. See U.S. Pat. No. 8,535,641; U.S. Patent Application Publication No. 2014/0030187 and Weichert, J. P., et al., Alkylphosphocholine analogs for broad-spectrum cancer imaging and therapy, Sci Transl Med, 2014, Jun. 11, 6(240), 240ra75; each of which are incorporated by reference herein in its entirety.

### SUMMARY

The present invention is directed to methods for the treatment of a cancer via the administering an effective amount of ¹³¹I-labeled 18-(p-iodo-phenyl)octadecyl phosphocholine or a salt thereof, wherein the ¹³¹I-labeled 18-(p-iodo-phenyl)octadecyl phosphocholine or a salt thereof is administered as a fractionated dose. The ¹³¹I-labeled 18-(p-iodo-phenyl)octadecyl phosphocholine or a salt thereof may be administered according to a single dosing cycle. The single dosing cycle may be a single dose once on day 1 and administering a single dose once on day 4 (3 days after day 1); administering a single dose once on day 1 and administering a single dose once on day 5 (4 days after day 1); administering a single dose once on day 1 and administering a single dose once on day 6 (5 days after day 1); administering a single dose once on day 1 and administering a single dose once on day 7 (6 days after day 1); administering a single dose once on day 1 and administering a single dose once on day 8 (7 days after day 1); administering a single dose once on day 1 and administering a single dose once on day 9 (8 days after day 1); administering a single dose once on day 1 and administering a single dose once on day 10 (9 days after day 1); administering a single dose once on day 1 and administering a single dose once on day 11 (10 days after day 1); administering a single dose once on day 1 and administering a single dose once on day 12 (11 days after day 1); and administering a single dose once on day 1 and administering a single dose once on day 13 (12 days after day 1). The single dosing cycle is administering a single dose once on day 1 and administering a single dose once on day 7 (6 days after day 1). Day 1 may be any day that the cycle begins. The subject may be a human. The ¹³¹I-labeled 18-(p-iodo-phenyl)octadecyl phosphocholine or a salt thereof is selective for cancer cells in the subject.

The ¹³¹I-labeled 18-(p-iodo-phenyl)octadecyl phosphocholine or a salt thereof may be administered at a dose range of from 1 mCi to 100 mCi per meter² ((m²). The dose may be 1 mCi to 100 mCi per kg body weight of the subject. The ¹³¹I-labeled 18-(p-iodo-phenyl)octadecyl phosphocholine or a salt thereof may be administered at a dose range of from 1 mCi to 100 mCi per meter² (m²) on day 1 and day 8 (7 days after day 1). The dose may be from 1 mCi to 100 mCi per kg body weight of the subject and administered on day 1 and day 8 (7 days after day 1). The cancer may be a multiple myeloma, lymphoma, neuroblastoma, adenocarcinoma, pediatric lymphoma, diffuse intrinsic pontine gliomas (DIPG), sarcoma, leukemia, metastatic tumor, a liver cancer, a lung cancer, a brain cancer, a pancreas cancer, a melanoma cancer, or a breast cancer. The cancer is amenable to fractionated dose radiation. The method may further another cancer therapy selected from the group consisting of chemotherapy, immunotherapy, cell therapy, radiosensitizing therapy, radioprotective therapy, external beam radiation, tumor resection, ablative therapy, and local physical treatment on the basis of cold (cryo), heat (thermal), radiofrequency, and microwave.

### BRIEF DESCRIPTION OF THE DRAWINGS

FIG. 1 shows individual tumor volume by dose group. FIG. 1A shows tumor volume over time after treatment with vehicle alone (saline). FIG. 1B shows tumor volume over time after treatment with a single dose of 50 μCi ¹³¹I-labeled 18-(p-iodo-phenyl)octadecyl phosphocholine. FIG. 1C shows tumor volume over time after treatment with a multi dose (days 1 and 8) of 50 μCi ¹³¹I-labeled 18-(p-iodo-phenyl)octadecyl phosphocholine. FIG. 1D shows tumor volume over time after treatment with a single dose of 100 μCi ¹³¹I-labeled 18-(p-iodo-phenyl)octadecyl phosphocholine. FIG. 1E shows tumor volume over time after treatment with a multi dose of Bortezomib (days 1, 4, 8, and 11).

FIG. 2 shows mean tumor volume over time after treatment with vehicle alone, a single dose of 50 μCi ¹³¹I-labeled 18-(p-iodo-phenyl)octadecyl phosphocholine, a multi dose (days 1 and 8) of 50 μCi ¹³¹I-labeled 18-(p-iodo-phenyl)octadeyl phosphocholine, a single dose of 100 μCi ¹³¹I-labeled 18-(p-iodo-phenyl)octadeyl phosphocholine, and a multi dose of Bortezomib (days 1, 4, 8, and 11).

FIG. 3 shows the survival of OPM-2 mouse models after treatment with vehicle alone, a single dose of 50 μCi ¹³¹I-labeled 18-(p-iodo-phenyl)octadecyl phosphocholine, a multi dose (days 1 and 8) of 50 μCi ¹³¹I-labeled 18-(p-iodo-phenyl)octadecyl phosphocholine, a single dose of 100 μCi ¹³¹I-labeled 18-(p-iodo-phenyl)octadecyl phosphocholine, and a multi dose of Bortezomib (days 1, 4, 8, and 11).

FIG. 4 shows mean body weight change of the OPM-2 mouse model of FIG. 3 after treatment with vehicle alone, a single dose of 50 μCi ¹³¹I-labeled 18-(p-iodo-phenyl)octadecyl phosphocholine, a multi dose (days 1 and 8) of 50 μCi ¹³¹I-labeled 18-(p-iodo-phenyl)octadecyl phosphocholine, a single dose of 100 μCi ¹³¹I-labeled 18-(p-iodo-phenyl)octadecyl phosphocholine, and a multi dose of Bortezomib (days 1, 4, 8, and 11).

FIG. 5 shows tumor doubling time after treatment with vehicle alone, a single dose of 50 μCi¹³¹I-labeled 18-(p-iodo-phenyl)octadecyl phosphocholine, a multi dose (days 1 and 8) of 50 μCi¹³¹I-labeled 18-(p-iodo-phenyl)octadecyl phosphocholine, a single dose of 100 μCi 131I-labeled 18-(p-iodo-phenyl)octadecyl phosphocholine, and a multi dose of Bortezomib (days 1, 4, 8, and 11).

FIG. 6 shows fractionated dosing of CLR 131 (¹³¹I-labeled 18-(p-iodo-phenyl)octadecyl phosphocholine) in an OPM-2 model system.

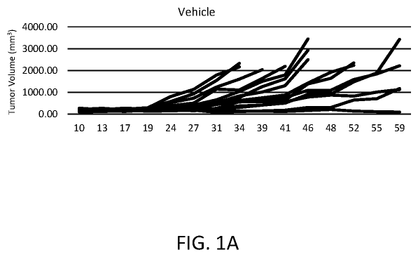

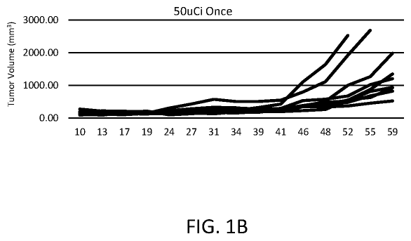

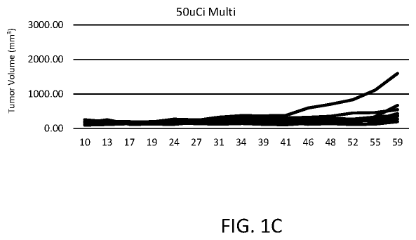

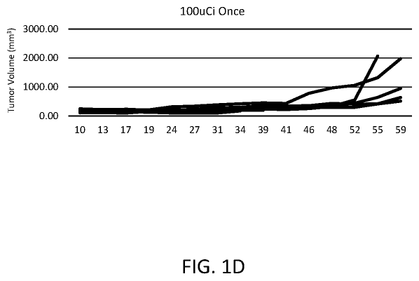

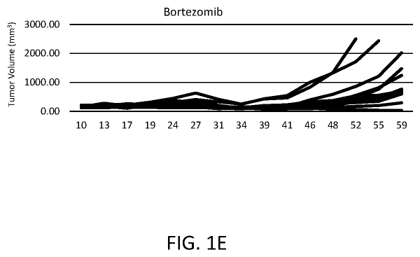

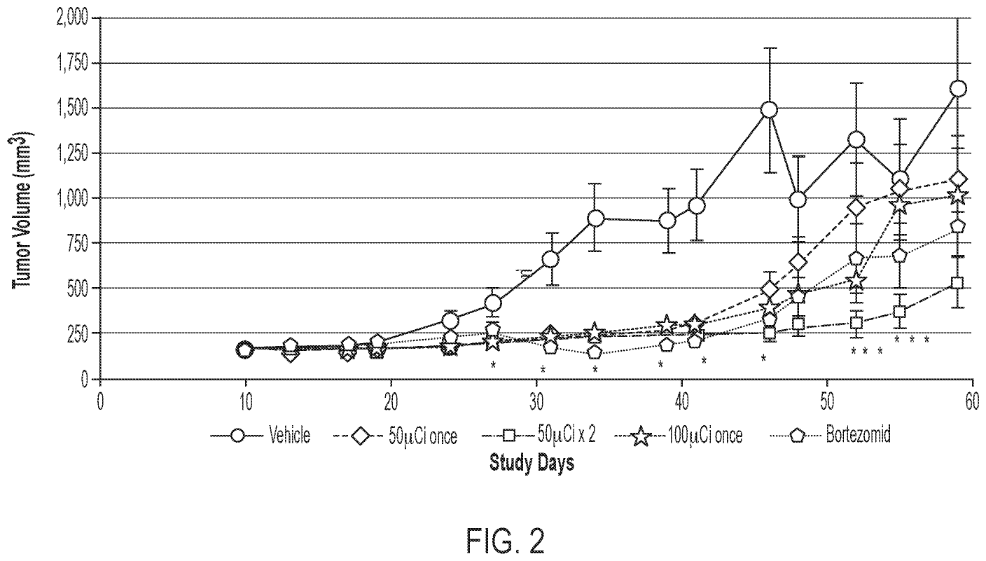

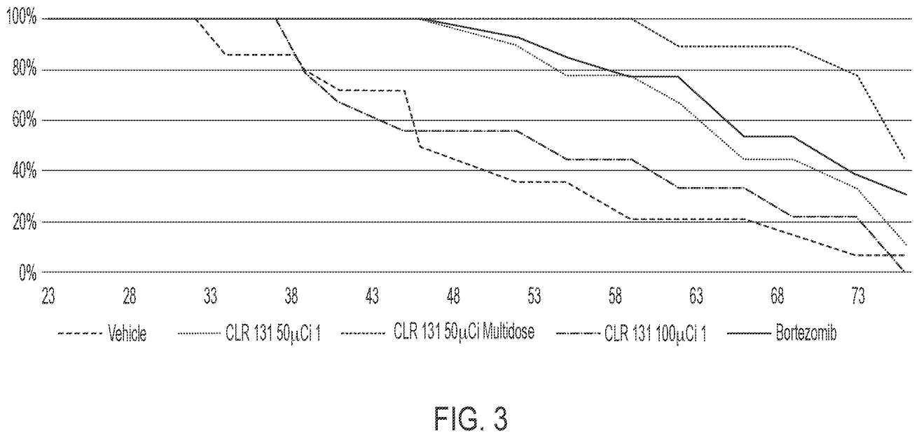

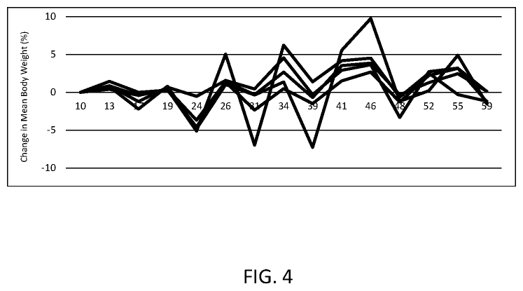

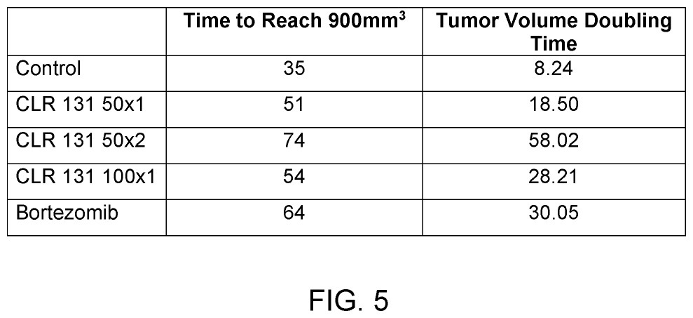

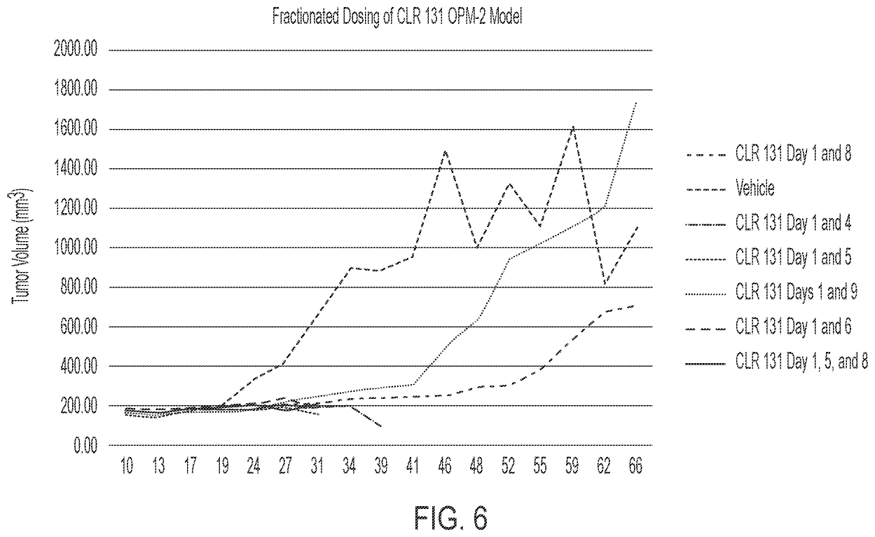

### DETAILED DESCRIPTION

The present disclosure provides a dosing regimen for the administration of CLR1404 for the treatment of cancer. As described herein, fractionated dosing of CLR1404 is well tolerated and is better tolerated than a single equal dose of CLR1404. Radioiodinated CLR1404, a second-generation phospholipid ether (“PLE”) analog is represented by the following structure:

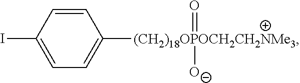

wherein I is a radiolabeled form of iodine (e.g. ¹²²I, ¹²³I, ¹²⁴I, ¹²⁵I, ¹³¹I, etc.). The present disclosure also provides methods for the treatment of cancer via a fractionated dosing regimen for CLR1404. As described herein, CLR1404 may be CLR131, which is ¹³¹I-labeled 18-(p-iodo-phenyl)octadecyl phosphocholine.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein, the term “treating” includes preventative as well as disorder remittent treatment including reducing, suppressing and inhibiting cancer progression or recurrence. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the terms “recurrence” and “recurrent” refer to the return of a disease after a remission.

As used herein, the term “administering” refers to bringing a patient, tissue, organ or cells in contact with an anti-cancer compound of the present invention. As used herein, administration can be accomplished in vitro (i.e., in a test tube) or in vivo, (i.e., in cells or tissues of living organisms, for example, humans). In certain embodiments, the present invention encompasses administering the compounds and/or compositions described herein to a patient or subject. A “patient” or “subject”, used equivalently herein, refers to a mammal, preferably a human, that either: (1) has a disorder remediable or treatable by administration of CLR1404 (e.g. CLR131 (¹³¹I-labeled 18-(p-iodo-phenyl)octadecyl phosphocholine (CLR131) or a salt thereof)), or (2) is susceptible to a disorder that is preventable by administering the CLR1404 (e.g. CLR131 (¹³¹I-labeled 18-(p-iodo-phenyl)octadecyl phosphocholine (CLR131) or a salt thereof)).

As used herein, the term “effective amount” refers to an amount sufficient to affect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful subject benefit. Thus, an “effective amount” will depend upon the context in which it is being administered. An effective amount may be administered in one or more prophylactic or therapeutic administrations.

As used herein the term “cancer” refers to any disease that results from the uncontrolled division of cells capable of metastasizing.

The term “malignant tumor cell” and “cancer cell” are used interchangeably throughout the specification. The term “malignant tumor stem cell” and “cancer stem cell” are used interchangeably throughout the specification.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Treat”, “treating” or “treatment” are each used interchangeably herein to describe reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of an antibody or pharmaceutical composition of the present invention to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, for example.

The invention includes the use of pharmaceutically acceptable salts of amino-substituted compounds with organic and inorganic acids, for example, citric acid and hydrochloric acid. Suitable pharmaceutically acceptable salts include, without limitation, acid addition salts which may, for example, be formed by mixing a solution of the alkylphosphocholine analog with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. The invention also includes N-oxides of the amino substituents of the compounds described herein. Pharmaceutically acceptable salts can also he prepared from the phenolic compounds by treatment with inorganic bases, for example, sodium hydroxide. Also, esters of the phenolic compounds can be made with aliphatic and aromatic carboxylic acids, for example, acetic acid and benzoic acid esters. As used herein, the term “pharmaceutically acceptable salt” refers to a compound formulated from a base compound which achieves substantially the same pharmaceutical effect as the base compound.

This invention further includes derivatives of the anti-cancer compounds. The term “derivatives” includes but is not limited to ether derivatives, acid derivatives, amide derivatives, ester derivatives and the like. In addition, this invention further includes methods utilizing hydrates of the anti-tumor compounds. The term “hydrate” includes but is not limited to hemihydrate, monohydrate, dihydrate, trihydrate and the like.

This invention further includes metabolites of the anti-cancer compounds. The term “metabolite” means any substance produced from another substance by metabolism or a metabolic process.

### 2. COMPOUNDS OF THE INVENTION

An exemplary compound of the invention is CLR1404, which is a radioiodinated phospholipid ether analog. The radioiodinated phospholipid ether analog may be ¹³¹I-labeled 18-(p-iodo-phenyl)octadecyl phosphocholine (CLR131) or a salt thereof. The basis for selective tumor targeting of the compounds of the present invention lies in differences between the plasma membranes of cancer cells as compared to those of most normal cells. Specifically, cancer cell membranes are highly enriched in “lipid rafts”. Cancer cells have five to ten times more lipid rafts than healthy cells. Lipid rafts are specialized regions of the membrane phospholipid bilayer that contain high concentrations of cholesterol and sphingolipids and serve to organize cell surface and intracellular signaling molecules (e.g., growth factor and cytokine receptors, the phosphatidylinositol 3-kinase (PI3K)/Akt survival pathway). Data suggests that lipid rafts serve as portals of entry for PLEs. The marked selectivity of these compounds for cancer cells versus non-cancer cells is attributed to the high affinity of PLEs for cholesterol and the abundance of .cholesterol-rich lipid rafts in cancer cells. The pivotal role played by lipid rafts is underscored by the fact that disruption of lipid raft architecture suppresses uptake of PLEs into cancer cells. It has been shown that the uptake of PLEs is reduced by 60% when lipid rafts are blocked from forming.

Results obtained in over 55 xenograft and spontaneous tumor models have universally shown the herein described compounds to undergo selective uptake and prolonged retention in tumors. Because the compound is metabolized to some extent in the liver, the inventors avoided earlier compound evaluation in liver tumor models due to high liver background radioactivity levels.

Results obtained in a variety of tumor models indicate that CLR1404, for example ¹³¹I-labeled 18-(p-iodo-phenyl)octadecyl phosphocholine, is sequestered and selectively retained by cancer cells and cancer stem cells. The herein described compounds have been shown to remain in cancer cells for up to 20 days. The compounds localize in both primary and metastatic lesions regardless of anatomic location including those found in lymph nodes.

### 3. COMPOSITIONS OF THE INVENTION

In another aspect, the present invention provides a pharmaceutical composition containing a compound of the present invention (e.g. ¹³¹I-labeled 18-(p-iodo-phenyl)octadecyl phosphocholine) in combination with one or more pharmaceutically acceptable carriers. In a preferred aspect the pharmaceutical composition is free of Kolliphor® EL (Kolliphor is a registered trademark of BASF SE). Kolliphor® EL is formerly known as Cremophor® EL (Cremophor is a registered trademark of BASF SE).

Actual dosage levels of active ingredients in the therapeutic compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated.

The present invention also provides pharmaceutical compositions that comprise compounds of the present invention formulated together with one or more pharmaceutically acceptable carriers. The pharmaceutical compositions can be specially formulated for oral administration in solid or liquid form, for parenteral administration or for rectal administration.

Any route of administration may be suitable for administering the herein described compositions and compounds to a subject. The compounds and compositions may be administered to the subject via intravenous injection. The disclosed compounds and compositions may be administered to the subject via any other suitable systemic deliveries, such as oral, parenteral, intranasal, sublingual, rectal, or transdermal. The compounds and compositions of this invention can be administered to subjects, such as humans and other mammals orally, rectally, parenterally, intracisternally, intravaginally, transdermally (e.g., using a patch), transmucosally, sublingually, pulmonary, intraperitoneally, topically (as by powders, ointments or drops), bucally or as an oral or nasal spray. The terms “parenteral” or “parenterally,” as used herein, refers to modes of administration which include intravenous, intramuscular, intraperitoneal, intrasternal, subcutaneous and intraarticular injection and infusion.

In another aspect, the present invention provides a pharmaceutical composition comprising a component of the present invention and a physiologically tolerable diluent. The present invention includes one or more compounds as described above formulated into compositions together with one or more physiologically tolerable or acceptable diluents, carriers, adjuvants or vehicles that are collectively referred to herein as diluents, for parenteral injection, for intranasal delivery, for oral administration in solid or liquid form, for rectal or topical administration, among others.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Injectable depot forms are made by forming microencapsule matrices of the compound or composition in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; (b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, com, germ, olive, castor and sesame oils), glycerol, tetrahydrofurfuryl alcohol, polyethylene glycols and fatty acid esters of sorbitan and mixtures thereof.

Besides inert diluents, any oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together. Methods to form liposomes are known in the art. See, for example, Prescott, Ed.; Methods in Cell Biology, Volume XIV, Academic Press, New York, N.Y. (1976), p. 33 et seq. Such compositions will influence the physical state, solubility, stability, rate of in vivo release, and rate of in vivo clearance.

In one method of the present invention, a pharmaceutical composition can be delivered in a controlled release system. For example, the compound or composition may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, for example liver, thus requiring only a fraction of the systemic dose (see, e.g., Goodson, in Medical Applications of Controlled Release, supra, vol. 2, pp. 115-P8 (1984). Other controlled release systems are discussed in the review by Langer (Science 249:1527-1533 (1990).

In another aspect, the invention is directed to a method of treating a disease or condition in a subject comprising administering to the subject an effective amount of a compound of the present invention.

In general, the invention is not limited to treatment of any specific disease or condition but encompasses the treatment of any disease or condition whose mechanism may be affected by the compounds of the present invention.

### 4. FRACTIONATED DOSING

In another aspect, the present invention provides a fractionated dosing regimen for the use of CLR1404 in the treatment of cancer. The CLR1404 may be ¹³¹I-labeled 18-(p-iodo-phenyl)octadecyl phosphocholine (CLR131). As used herein “fraction” or “fractionation” of radiation therapy is the division of the total dose of radiation therapy into several smaller doses delivered over a period of time. The total dosage may be fractionated, for example, to allow normal cells time to recover, to allow tumor cells that were in a relatively radio-resistant phase of the cell cycle during one treatment to cycle into a sensitive phase of the cycle before the next fraction is given, or to allow hypoxic tumor cells to reoxygenate between fractions, improving the tumor cell kill. The summed value of individual fractionized dose may add up to about the total dose of radiation therapy prescribed.

The dose may be administered on a per meter² (m²) or a per weight (e.g. kg) basis. The ¹³¹I-labeled 18-(p-iodo-phenyl)octadecyl phosphocholine or a salt thereof may be administered at a dose range of from 1 mCi to 100 mCi per meter² (m²); from 5 mCi to 90 mCi per meter² (m²); from 10 mCi to 80 mCi per meter² (m²); from 20 mCi to 70 mCi per meter² (m²); from 30 mCi to 60 mCi per meter² (m²); or from 40 mCi to 50 mCi per meter² (m²), for example. The dose may be 10 mCi per meter² (m²), 20 mCi per meter² (m²), 30 mCi per meter² (m²), 40 mCi per meter² (m²), 50 mCi per meter² (m²), 60 mCi per meter² (m²), 70, mCi per meter² (m²), 80 mCi per meter² (m²), 90 mCi per meter² ((m²), or 100 mCi per meter² (m²), for example.

The ¹³¹I-labeled 18-(p-iodo-phenyl)octadecyl phosphocholine or a salt thereof may be administered at a dose range of from 1 mCi to 100 mCi per kg; from 5 mCi to 90 mCi per kg; from 10 mCi to 80 mCi per kg; from 20 mCi to 70 mCi per kg; from 30 mCi to 60 mCi per kg; or from 40 mCi to 50 mCi per kg, for example. The dose may be 10 mCi per kg, 20 mCi per kg, 30 mCi per kg, 40 mCi per kg, 50 mCi per kg, 60 mCi per kg, 70, mCi per kg, 80 mCi per kg, 90 mCi per kg, or 100 mCi per kg, for example.

In one aspect, the compounds or compositions are administered to a subject in need thereof as fractionated doses in two or more fractions. In another embodiment, the compounds or compositions are administered as fractionated doses in a hyperfractionation therapy. In another aspect, the compounds or compositions are administered as fractionated doses in an accelerated fractionation therapy.

In certain embodiments, the methods and compositions of the present disclosure are useful for treating cancer. The delivery modality/regimen may include, for example, conventional fractionation therapy, hyperfractionation, hypofractionation, and accelerated fractionation.

In one embodiment, the therapeutic regimen is hyperfractionation therapy. In hyperfractionation, higher tumor doses are delivered while maintaining a level of long-term tissue damage that is clinically acceptable. The daily dose is unchanged or slightly increased while the dose per fraction is decreased, and the overall treatment time remains constant.

In one embodiment, the therapeutic regimen is accelerated fractionation therapy. In the accelerated fractionation therapy, the dose per fraction is unchanged while the daily dose is increased, and the total time for the treatment is reduced.

In one embodiment, the therapeutic regimen is continuous hyperfractionated accelerated radiation therapy (CHART) therapy. In (CHART) therapy, an intense schedule of treatment in which multiple daily fractions are administered within an abbreviated period.

Fractionated doses of radiation therapy may be administered at intervals. In certain embodiments, the fractionized doses are administered over a period of minutes, hours, or weeks such as 1 to 26 weeks, such as from about 1 to 15 weeks, such as from 2 to 12 weeks. In certain embodiments, the fractionized doses are administered over a period less than about 15 weeks, such as less than about 14 weeks such as less than about 13 weeks, such as less than about 12 weeks, such as less than about 11 weeks, such as about less than about 10 weeks, such as less than about 9 weeks, such as less than about 8 weeks, such as less than about 7 weeks, such as less than about 6 weeks, such as less than about 5 weeks, such as less than about 4 weeks. In certain embodiments, the cumulative external irradiation is a therapeutically effective amount of radiation for killing cells. The fractionated dose for CLR1404 (e.g. CLR131, ¹³¹I-labeled 18-(p-iodo-phenyl)octadecyl phosphocholine) may be administered as a single dose once on day 1 and again as a single dose 7 days later (day 8). The fractionated dose for CLR1404 (e.g. CLR131, ¹³¹-labeled 18-(p-iodo-phenyl)octadecyl phosphocholine) that is administered as a single dose once on day 1, and again as a single dose 7 days later, may be a single dosing cycle. The fractionated dose for CLR1404 (e.g. CLR131, ¹³¹I-labeled 18-(p-iodo-phenyl)octadecyl phosphocholine) may be administered as a single dose once on day 1 and again as a single dose 4 days later (day 5). The fractionated dose for CLR1404 (e.g. CLR131, ¹³¹I-labeled 18-(p-iodo-phenyl)octadecyl phosphocholine) may be administered as a single dose once on day 1 and again as a single dose 5 days later (day 6). The fractionated dose for CLR1404 (e.g. CLR131, ¹³¹I-labeled 18-(p-iodo-phenyl)octadecyl phosphocholine) may be administered as a single dose once on day 1 and again as a single dose 6 days later (day 7). The fractionated dose for CLR1404 (e.g. CLR131, ¹³¹I-labeled 18-(p-iodo-phenyl)octadecyl phosphocholine) may be administered as a single dose once on day 1 and again as a single dose 8 days later (day 9). The fractionated dose for CLR1404 (e.g. CLR131, ¹³¹I-labeled 18-(p-iodo-phenyl)octadecyl phosphocholine) may be administered as a single dose once on day 1 and again as a single dose 9 days later (day 10). The fractionated dose for CLR1404 (e.g. CLR131, ¹³¹I-labeled 18-(p-iodo-phenyl)octadecyl phosphocholine) may be administered as a single dose once on day 1 and again as a single dose 10 days later (day 11). The fractionated dose for CLR1404 (e.g. CLR131, ¹³¹I-labeled 18-(p-iodo-phenyl)octadecyl phosphocholine) may be administered as a single dose once on day 1 and again as a single dose 11 days later (day 12). The fractionated dose for CLR1404 (e.g. CLR131, ¹³¹I-labeled 18-(p-iodo-phenyl)octadecyl phosphocholine) may be administered as a single dose once on day 1 and again as a single dose 12 days later (day 13). The fractionated dose for CLR1404 (e.g. CLR131, ¹³¹I-labeled 18-(p-iodo-phenyl)octadecyl phosphocholine) may be administered as a single dose once on day 1 and again as a single dose 13 days later (day 14). Day 1 may be the first day of treatment with CLR1404 (e.g. CLR131, ¹³¹I-labeled 18-(p-iodo-phenyl)octadecyl phosphocholine).

a. Combination Therapy

A number of chemotherapeutic agents may enhance the effects of the CLR1404-based radiation therapy, for example ¹³¹I-labeled 18-(p-iodo-phenyl)octadecyl phosphocholine-based therapy, as described herein. In one aspect, the aspects and embodiments of the present disclosure can be utilized as a combined therapy with existing chemotherapeutic modalities. The combination (sequential or concurrent) therapy can be co-administration or co-formulation.

In those embodiments where the patient is subjected to more than one form of radiation therapy, for example, in addition to the CLR1404-based therapy, the patient may be subjected to one or more additional forms of radiation therapy at the same time, in sequence, in fractional doses at the same time or in fractional doses sequentially, in fractional doses alternating, and/or any combination thereof. In certain embodiments, intraoperative radiation therapy is administered before, during and/or after a surgical procedure and a second form or radiation therapy is administered at a later time such as hours after the surgical procedure, and/or days after the surgical procedure, and/or weeks after the surgical procedure. The intraoperative radiation and/or the second form of radiation may be CLR-1404-based (e.g. ¹³¹I-labeled 18-(p-iodo-phenyl)octadecyl phosphocholine or a salt thereof). In certain embodiments, the patient is treated with radiation therapy, for example, CLR-1404-based therapy (e.g. ¹³¹I-labeled 18-(p-iodo-phenyl)octadecyl phosphocholine or a salt thereof), leading up to the surgical procedure such as hours before the surgical procedure, days before the surgical procedure and/or weeks before the surgical procedure.

The CLR1404-based (e.g. CLR131, ¹³¹I-labeled 18-(p-iodo-phenyl)octadecyl phosphocholine) radiation therapy may be combined with one or more other therapies, wherein the reduction of the cancer with the CLR1404 therapy takes place concurrently, subsequently, or prior to another treatment. For example, the other treatment may be radiotherapy, chemotherapy, tumor resection, ablative therapy, and/or local physical treatment on the basis of cold (cryo), heat (thermal), radiofrequence, and microwave. The CLR1404-based radiation therapy may be used in conjunction with hyperthermia, i.e., the use of heat. In certain embodiments, the combination of heat and radiation can increase the response rate of some tumors.

In combination with the CLR-1404-based (e.g. CLR131, ¹³¹I-labeled 18-(p-iodo-phenyl)octadecyl phosphocholine) radiation therapy, a radiosensitizer may be administered in conjunction with an additional agent. For example, a nitroimidazole may be administered in conjunction with an additional agent such as a targeting agent, a chemotherapeutic agent or a second radiosensitizer. Targeting agents include any suitable agents for targeting cancer cells, such as antibodies. Nitroimidazole may be bound to the targeting agent through covalent or non-covalent attachments. For example, nitroimidazoles, such as 2-nitroimidazoles, may be bound to a targeting agent through a linker such as a biodegradable linker. Alternatively, the nitroimidazoles may be bound to a targeting agent through ionic interactions. In certain embodiments, the radiosensitizer of the invention and the additional agent may be enveloped within a liposome. These chemotherapeutic agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxanes (paclitaxel, docetaxel), vincristine, vinblastine, nocodazole, epothilones, and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamine, oxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates (busulfan), nitrosoureas (carmustine (BCNU) and analogs, streptozocin), trazenes (e.g., dacarbazinine (DTIC)); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; chromatin disruptors.

Radioprotectors may be administered to a patient in combination with the methods described herein. Radioprotectors, also called radioprotectants, are drugs that protect normal (noncancerous) cells from the damage caused by radiation therapy. These agents promote the repair of normal cells that are exposed to radiation. Exemplary radioprotectants include Amifostine.

In certain embodiments, the methods of the invention further comprise administration of a bacterium such as salmonella or genetically engineered variants thereof. Studies have shown that the combination of radiation therapy with salmonella increases the effectiveness of tumor suppression particularly in the presence of inflammatory cells called neutrophils. Therapies which combine a nitroimidazoles with a bacterium such as salmonella and CLR1404-based radiotherapy may enhance tumor suppression.

Radiation sensitizers of the invention may be formulated in a conventional manner using one or more physiologically acceptable carriers or excipients. For example, compounds of the invention and their physiologically acceptable salts and solvates may be formulated for administration by, for example, injection (e.g. subcutaneous, intramuscular, intraparenteral), inhalation or insufflation (either through the mouth or the nose) or oral, buccal, sublingual, transdermal, nasal, parenteral or rectal administration. In one embodiment, a compound of the invention may be administered locally, at the site where the tumor cells are present, i.e., in a specific tissue, organ, or fluid (e.g., blood, cerebrospinal fluid, etc.). Typically, compounds and compositions of the invention are administered intravenously.

b. Cancers

A cancer that can be treated with compounds and compositions of the present invention may be any cancer. Cancers that can be treated with the compounds and compositions as described herein include, but are not limited to: multiple myeloma, lymphoma, metastatic cancer, sarcoma, neuroblastoma, leukemia, breast cancer including male breast cancer; adenocarcinomas, diffuse intrinsic pontine gliomas (DIPG), pediatric lymphoma, digestive/gastrointestinal cancers including anal cancer, appendix cancer, extrahepatic bile duct cancer, gastrointestinal carcinoid tumor, colon cancer, esophageal cancer, gallbladder cancer, gastric cancer, gastrointestinal stromal tumors (“gist”), Islet cell tumors, adult primary liver cancer, childhood liver cancer, pancreatic cancer, rectal cancer, small intestine cancer, and stomach (gastric) cancer; endocrine and neuroendocrine cancers including pancreatic adenocarcinoma, adrenocortical carcinoma, pancreatic neuroendocrine tumors, Merkel cell carcinoma, non-small cell lung neuroendocrine tumor, small cell lung neuroendocrine tumor, parathyroid cancer, pheochromocytoma, pituitary tumor and thyroid cancer; eye cancers including intraocular melanoma and retinoblastoma; genitourinary cancer including bladder cancer, kidney (renal cell) cancer, penile cancer, prostate cancer, transitional cell renal pelvis and ureter cancer, testicular cancer, urethral cancer and Wilms tumor; germ cell cancers including childhood central nervous system cancer, childhood extracranial germ cell tumor, extragonadal germ cell tumor, ovarian germ cell tumor and testicular cancer; gynecologic cancers including cervical cancer, endometrial cancer, gestational trophoblastic tumor, ovarian epithelial cancer, ovarian germ cell tumor, uterine sarcoma, vaginal cancer and vulvar cancer; head and neck cancers including hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, parathyroid cancer, pharyngeal cancer, salivary gland cancer and throat cancer; leukemias including adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; lymphomas including AIDS-related lymphoma, cutaneous t-cell lymphoma, adult Hodgkin lymphoma, childhood Hodgkin lymphoma, Hodgkin lymphoma during pregnancy, mycosis fungoides, adult non-Hodgkin lymphoma, childhood non-Hodgkin lymphoma, non-Hodgkin lymphoma during pregnancy, primary central nervous system lymphoma, Sezary syndrome and Waldenstrom macroglobulinemia; musculoskeletal cancers including Ewing sarcoma, osteosarcoma and malignant fibrous histocytoma of bone, childhood rhabdomyosarcoma and soft-tissue sarcoma; neurological cancers including adult brain tumor, childhood brain tumor, astrocytomas, brain stem glioma, central nervous system atypical teratoid/rhabdoid tumor, central nervous system embryonal tumors, craniopharyngioma, ependymoma, neuroblastoma, primary central nervous system (CNS) lymphoma; respiratory/thoracic cancers including non-small cell lung cancer, small cell lung cancer, malignant mesothelioma, thymoma and thymic carcinoma; and skin cancers including Kaposi sarcoma, melanoma and squamous cell carcinoma. Multiple myeloma (MM) is a type of cancer that occurs in circulating blood cells (i.e. plasma B cells), rather than in solid tissue. Multiple myeloma is a universally fatal disease, comprising 15% of hematological malignancies and 1% of all cancers. It has a median survival of 5-7 years from diagnosis. While newer drugs, such as bortezomib and lenalidomide, have exhibited increased response to therapy, patients inevitably relapse and become resistant to therapy.

### 5. EXAMPLES

The foregoing may be better understood by reference to the following examples, which are presented for purposes of illustration and are not intended to limit the scope of the invention. The present invention has multiple aspects, illustrated by the following non-limiting examples.

### Example 1

The OPM-2 cell line (human multiple myeloma) was purchased from American Type Culture Collection (ATCC, Rockville, Md.) and maintained in McCoy's 5a media supplemented with 10% fetal bovine serum. Female CB17 SCID mice approximately 5-7 weeks of age were injected subcutaneously with 1×10⁷ viable cells (in ˜100 μL Dulbecco's PBS) into the right flank. The study was initiated when tumor size had reached a pre-determined size (approximately 150-200 mm3). The mice were given potassium iodide at a concentration of 0.1% in their drinking water to block possible free iodide in the drug formulation three days prior to injection and continuing through two weeks post-injection. Mice were randomly assigned to dose groups. Tumor volume was measured with a caliper during the course of the study. Tumor doubling time calculation4 TDT=D×log(2)/log(1+r/100), where D equals days between measurements and r=rate of growth; r/100=(V2−V1/V1)×100%. Statistical analysis: 1-way Anova, Dunnett's test.

Table 1. Dosing Regimen.

TABLE 1

| | | | Dose Volume | |
|---|---|---|---|---|
| | Dose | | (μL) | Dosing Days |
| Vehicle (n = 14) | 0 | mg/kg | 100 | 1 and 8 |
| CLR 131 (n = 9) | 50 | μCi | 100 | 1 |
| CLR 131 (n = 9) | 50 | μCi | 100 | 1 and 8 |
| CLR 131 (n = 9) | 100 | μCi | 100 | 1 |
| Bortezomib (n = 14) | 0.6 | mg/kg | 100 | 1, 4, 8, 11 |

### Example 2

Repeated/fractionated dosing of CLR 131 was well tolerated and better tolerated than the single equal dose. All doses of CLR 131 showed marked antitumor activity in this model of multiple myeloma. Single dose infusions result in similar inhibition of MM similar to bortezomib. Fractionated dosing results in statistically significant reduction in tumor volume vs control after day 26. Fractionated dosing results in statistically significant reduction in tumor volume vs all other treatments at day 52 (p<0.05). Tumor doubling time was markedly increased for fractionated dosing versus all other treatments. Fractionated dosing resulted in a statistically significant survival benefit.

### Example 3

As demonstrated in Table 2, fractionated dosing (Cohort 5) provided 18% more drug, as measured by the actual dose of millicuries delivered to the patient than single bolus dose (Cohort 4). However even though more drug was provided to the patient the average grade of adverse events reduced and the median grade remained the same. Additionally, efficacy assessments also showed improvement between the single bolus dose and the fractionated dosing regimen. Median overall survival increased from 6.5 months (bolus dose) to 7.4 months (fractionated dose) with assessment of the median overall survival in the fractionated dose group still ongoing. Average progression free survival increased from 2.8 to 2.9 months, respectively, again with assessment ongoing in the fractionated dose group. Average reduction in surrogate markers of efficacy were greater between the cohorts with patients receiving a single bolus dose experience on average a 29% reduction in surrogate markers versus an average 40% reduction for those receiving a fractionated dose.

Dosing is based upon body surface area or BSA in millicuries/meter squared (mCi/m²). Patients either received a single 30 minute infusion of 31.25 mCi/m² on day 1 or a 30 minute fractionated infusion dose of 15.625 mCi/m² on day 1 and then repeated on day 8.

Table 2. Fractionated Dose Outcomes in Clinical Trials.

TABLE 2

| | Total mCi | Average | Median |
|---|---|---|---|
| Adverse Events | Dosed | Grade | Grade |
| Cohort 4 (31.25 mCi/m²) | 55.29 | 3.23 ± 0.93 | 3.0 |
| CLR 131 (n = 9) | 65.15 | 2.95 ± 1.10 | 3.0 |

For reasons of completeness, various aspects of the invention are set out in the following numbered clause:

Clause 1. A method for the treatment of a cancer in a subject comprising:

a) administering an effective amount of ¹³¹I-labeled 18-(p-iodo-phenyl)octadecyl phosphocholine or a salt thereof, wherein the ¹³¹I-labeled 18-(p-iodo-phenyl)octadecyl phosphocholine or a salt thereof is administered as a fractionated dose.

Clause 2. The method of clause 1, wherein the ¹³I-labeled 18-(p-iodo-phenyl)octadecyl phosphocholine or a salt thereof is administered pursuant to a single dosing cycle selected from the group consisting of administering a single dose once on day 1 and administering a single dose once on day 4, administering a single dose once on day 1 and administering a single dose once on day 5, administering a single dose once on day 1 and administering a single dose once on day 6, administering a single dose once on day 1 and administering a single dose once on day 7, administering a single dose once on day 1 and administering a single dose once on day 8, administering a single dose once on day 1 and administering a single dose once on day 9, administering a single dose once on day 1 and administering a single dose once on day 10, administering a single dose once on day 1 and administering a single dose once on day 11, administering a single dose once on day 1 and administering a single dose once on day 12, and administering a single dose once on day 1 and administering a single dose once on day 13.

Clause 3. The method of clause 2, wherein the single dosing cycle is administering a single dose once on day 1 and administering a single dose once on day 8 (7 days after day 1).

Clause 4. The method of clause 1, wherein the subject is a human.

Clause 5. The method of clause 1, wherein the ¹³I-labeled 18-(p-iodo-phenyl)octadecyl phosphocholine or a salt thereof is selective for cancer cells in the subject.

Clause 6. The method of clause 1, wherein the ¹³I-labeled 18-(p-iodo-phenyl)octadecyl phosphocholine or a salt thereof is administered at a dose range of from 1 mCi to 100 mCi per meter² (m²) on day 1 and day 8 (7 days after day 1).

Clause 7. The method of clause 1, wherein the dose is 1 mCi to 100 mCi per kg body weight of the subject.

Clause 8. The method of clause 1, wherein the cancer is a multiple myeloma, lymphoma, neuroblastoma, sarcoma, leukemia, metastatic tumor, a liver cancer, a lung cancer, a brain cancer, a pancreas cancer, a melanoma cancer, adenocarcinoma, pediatric lymphoma, diffuse intrinsic pontine gliomas (DIPG), or a breast cancer.

Clause 9. The method of clause 1, wherein the cancer is amenable to fractionated dose radiation.

Clause 10. The method of clause 1, wherein the method further comprises another cancer therapy selected from the group consisting of chemotherapy, immunotherapy, cell therapy, radiosensitizing therapy, radioprotective therapy, external beam radiation, tumor resection, ablative therapy, and local physical treatment on the basis of cold (cryo), heat (thermal), radiofrequency, and microwave.

Clause 11. The method of clause 6, wherein the ¹³¹I-labeled 18-(p-iodo-phenyl)octadecyl phosphocholine or a salt thereof is administered at a dose range of from 1 mCi to 100 mCi per meter² (m²) on day 1 as a single dose and day 8 as a single dose (7 days after day 1).

Clause 12. The method of clause 7, wherein the dose is 1 mCi to 100 mCi per kg body weight of the subject on day 1 as a single dose and day 8 as a single dose (7 days after day 1).

What is claimed is:

1. A method for the treatment of a cancer in a subject comprising:
a) administering an effective amount of ¹³¹I-labeled 18-(p-iodo-phenyl)octadecyl phosphocholine or a salt thereof, wherein the ¹³¹I-labeled 18-(p-iodo-phenyl)octadecyl phosphocholine or a salt thereof is administered as a fractionated dose.

2. The method of claim 1, wherein the ¹³¹I-labeled 18-(p-iodo-phenyl)octadecyl phosphocholine or a salt thereof is administered pursuant to a single dosing cycle selected from the group consisting of administering a single dose once on day 1 and administering a single dose once on day 4, administering a single dose once on day 1 and administering a single dose once on day 5, administering a single dose once on day 1 and administering a single dose once on day 6, administering a single dose once on day 1 and administering a single dose once on day 7, administering a single dose once on day 1 and administering a single dose once on day 8, administering a single dose once on day 1 and administering a single dose once on day 9, administering a single dose once on day 1 and administering a single dose once on day 10, administering a single dose once on day 1 and administering a single dose once on day 11, administering a single dose once on day 1 and administering a single dose once on day 12, and administering a single dose once on day 1 and administering a single dose once on day 13.

3. The method of claim 2, wherein the single dosing cycle is administering a single dose once on day 1 and administering a single dose once on day 8 (7 days after day 1).

4. The method of claim 1, wherein the subject is a human.

5. The method of claim 1, wherein the ¹³¹I-labeled 18-(p-iodo-phenyl)octadecyl phosphocholine or a salt thereof is selective for cancer cells in the subject.

6. The method of claim 1, wherein the ¹³¹I-labeled 18-(p-iodo-phenyl)octadecyl phosphocholine or a salt thereof is administered at a dose range of from 1 mCi to 100 mCi per meter² (m²).

7. The method of claim 1, wherein the dose is 1 mCi to 100 mCi per kg body weight of the subject.

8. The method of claim 1, wherein the cancer is a multiple myeloma, lymphoma, neuroblastoma, sarcoma, leukemia, metastatic tumor, a liver cancer, a lung cancer, a brain cancer, a pancreas cancer, a melanoma cancer, adenocarcinoma, DIPG, pediatric lymphoma, or a breast cancer.

9. The method of claim 1, wherein the cancer is amenable to fractionated dose radiation.

10. The method of claim 1, wherein the method further comprises another cancer therapy selected from the group consisting of chemotherapy, immunotherapy, cell therapy, radiosensitizing therapy, radioprotective therapy, external beam radiation, tumor resection, ablative therapy, and local physical treatment on the basis of cold (cryo), heat (thermal), radiofrequency, and microwave.

11. The method of claim 1, wherein the ¹³¹I-labeled 18-(p-iodo-phenyl)octadecyl phosphocholine or a salt thereof is administered at a dose range of from 1 mCi to 100 mCi per meter² (m²) on day 1 as a single dose and day 8 as a single dose (7 days after day 1).

12. The method of claim 7, wherein the dose is 1 mCi to 100 mCi per kg body weight of the subject and administered on day 1 as a single dose and day 8 as a single dose (7 days after day 1).
